(19) United States
(12) Patent Application Publication
(10) Pub. No.: US 2009/0176220 A1
(43) Pub. Date: Jul. 9, 2009

(54) ANTIBODY CHARACTERIZATION TEST

(75) Inventors: Frederic Dhainaut, Boissy Le Sec (FR); Jean-Luc Teillaud, Paris (FR); Laurent Siret, Villiers Sur Orge (FR)

Correspondence Address: MARSH, FISCHMANN & BREYFOGLE LLP, 8055 East Tufts Avenue, Suite 450, Denver, CO, 80237, US

(73) Assignee: LFB Biotechnologies, Les Ulis (FR)

(21) Appl. No.: 12/097,084

(22) PCT Filed: Dec. 15, 2006
(86) PCT No.: PCT/FR2006/002744

§ 371 Date: Dec. 12, 2008

(30) Foreign Application Priority Data
Dec. 16, 2005 (FR) 0512813

(51) Int. Cl. C12Q 1/68; C12Q 1/02

(52) U.S. Cl. 435/6; 435/29

(57) ABSTRACT

The present invention relates to a method for measuring the ability of an antibody preparation to activate an Fc receptor, wherein this method comprises the following steps: a) aggregating said antibodies with one another, b) bringing cells expressing an Fc receptor into contact with said aggregated antibodies, and c) measuring the reaction of the cells resulting from the activation of the Fc receptor of said cells by the Fc region of said antibodies.

### INTRODUCTION AND PRIOR ART

The present invention relates to a method for measuring the ability of an antibody preparation to activate an Fc receptor, this method comprising the following stages:

a) aggregating said antibodies with each other,

b) bringing cells expressing an Fc receptor into contact with said aggregated antibodies, and

c) measuring the reaction of the cells resulting from the activation of the Fc receptor of said cells by the Fc region of said antibodies.

Increasingly used in research, antibodies also constitute the tools of choice in diagnostics and in therapeutics, where they are an alternative to conventional treatments.

Numerous antibody preparations for therapeutic use, of plasmatic or biotechnological origin, are currently on the market, or in the clinical development phase. Their properties are exploited in order to obtain therapeutic tools capable of binding specifically to their target, and effectively recruiting immune cells.

These last few years, research has been directed towards improving the effectiveness of antibodies, and more particularly towards the manipulation of their constant Fc region. It is the latter which is responsible for the “effector” properties of the antibodies, as it allows the mobilization of the effector immune cells and complement molecules. This ability is made possible by the presence, on certain immune cells, of glycoproteins, the Fc receptors or FcRs. These receptors are capable of binding to the constant region of the antibodies, once the latter have bound, by their variable region, the target antigen. On contact with these cells, the antibodies trigger different cell mechanisms such as phagocytosis and ADCC (Antibody-Dependent Cell-mediated Cytotoxicity).

This poses the question of knowing how to discriminate between antibodies on the basis of their “effectiveness”, i.e. their ability to trigger the immune cell mechanisms, due to the binding of the Fc region of the antibodies to the Fc receptors.

In document FR 02 11416, the Applicant describes a method for measuring the functional activity of an antibody. This method consists of bringing cells expressing the CD16 receptor into contact in a reaction medium, in the presence of the antibody and the antigen of said antibody, and measuring the quantity of at least one cytokine produced by the cell expressing the CD16 receptor, this measurement representing the activation of the effector cell of the immune system by the antibody.

Thus, this method makes it possible to evaluate the activation of effector cells by the antigen-antibody complex. However, in this method, the activation of the effector cells is both dependant on the affinity of the antibody for its target and on the ability of the Fc region to bind to the Fc receptor. This test therefore does not make it possible to evaluate the activation of the effector cells due to the single Fc region of the antibody.

In order to respond to this problem, the document EP 1 298 219 proposes a method making it possible to measure the functionality of the Fc region of an antibody, without this measurement being influenced by the binding ability of the Fab region of the antibody. This method consists of immobilizing the antibody to be tested, introducing it into the presence of effector cells expressing Fc receptors, and measuring the reaction in the effector cell resulting from the activation of the effector cell by the Fc region of the antibody. In this method, the antibodies are immobilized by coating on a plate or beads.

The number of antibodies thus immobilized cannot be controlled, as it depends both on the correct binding of the antibodies to the plate, their orientation on the plate or beads, and the electrical charge of the antibody. In fact, the electrical charge of the antibodies depends of their level of amination, which affects their ability to become immobilized on beads or plates. Thus, this parameter causes a variability of the test with as a function of the antibodies to be tested, thus making it difficult to compare the functionality of the Fc region of two antibodies of different sequence and/or specificity. Furthermore, the test is carried out using a human monocyte line (THP-1) which must be activated by interferon γ for consistent surface expression of the FcγRIIIa receptor. This prior activation is a significant source of variability of the test and therefore it is difficult to compare experiments.

Thus, the methods of the state of the art proposing to discriminate between antibodies on the basis of their “effectiveness”, are either not very suitable for an evaluation of the activation of the effector cells due to the Fc region of the antibodies, or not very reproducible, and therefore difficult to standardize. This is why it was the intention of the Applicant to develop a novel method, making it possible to measure the ability of an antibody preparation to activate an Fc receptor which would both be reproducible, and specifically allow evaluation of the ability of the Fc region of an antibody to activate an Fc receptor without the characteristics of the Fab region of the antibody interfering in this evaluation.

### DETAILED DESCRIPTION OF THE INVENTION

Thus a first object of the invention relates to a method for measuring the ability of an antibody preparation to activate an Fc receptor, comprising the following steps:

a) aggregating said antibodies with each other,

b) bringing cells expressing an Fc receptor into contact with said aggregated antibodies, and

c) measuring the reaction of the cells resulting from the activation of the Fc receptor of said cells by the Fc region of said antibodies.

For the purposes of the invention, by “aggregating said antibodies with each other” is meant combining together antibodies dispersed in the solution containing them, to form a network expressing strong coupling between the antibodies of the network.

This aggregation of the antibodies has the function of orientating the antibodies in a controlled and homogeneous fashion, in the direction suited to a suitable presentation of the Fc region of all the antibodies or a large majority thereof towards the Fc receptor of the effector cells expressing an Fc receptor. In fact, the Applicant has surprisingly noted that the effect of such an aggregation is the fact that the Fc regions of certain antibodies are oriented in a particular fashion regardless of the specificity or the primary sequence of the antibodies studied.

The method according to the invention has the advantage of allowing the study of the dose-response relationship between the quantity of antibodies utilized in the method and the activation of the effector cell, and of being reproducible.

Furthermore, the Applicant has surprisingly noted that such a method allows an activation of the effector cells via the Fc receptors, without the presence of a target antigen. In fact, the Fc receptors are capable, in vivo of binding to the constant region of the antibodies, once the latter have bound, by their variable region, the target antigen. The absence of cells expressing the antigen target at the surface has the major advantage of dispensing with the presence of these cells, the source of variability in the biological tests, and therefore reducing the parameters capable of introducing a variability into the use of the method.

Thus, the biological variability due to the presence of target cells in the methods of the prior art as well as the influence of specificity and of the primary sequence of the antibody is limited, or even zero, in the method of the invention.

For implementing the invention, the antibodies can be aggregated using all the means known to a person skilled in the art for aggregating antibodies, such as heat or immunological tools, for example entire immunoglobulins G, this list being in no way limitative. Moreover, the method according to the invention has the advantage of being implemented with antibodies in solution.

For the purposes of the invention, by “Fc receptor” is meant any receptor of the Fc region of the antibodies, present on the effector cells, such as CD16 (FcgammaRIII and CD32 (FcgammaRII).

For the purposes of the invention, by “antibody” is meant any antibody, whatever its specificity and its isotype, provided that it comprises an Fc region or a region possessing the same functions as the Fc region. Thus, it can be a whole antibody or an antibody fragment, for example an Fc antibody fragment. Moreover, the antibodies utilized in the method according to the invention can be IgGs (IgG1 or IgG2 or IgG3 or IgG4) IgMs, IgEs, IgAs or IgDs, or also a mixture of these. Moreover, the antibodies utilized in the method of the invention can be monoclonal and/or polyclonal. In the case where they are monoclonal antibodies, these antibodies can be chimeric, humanized, human or of animal origin. For the purposes of the invention, by “cell expressing an Fc receptor” is meant any cell expressing at its surface an Fc receptor, this cell being capable of expressing such a receptor naturally or following a genetic modification. By way of example, mention can be made of NK cells, activated monocytes, peripheral blood granulocytes, macrophages, neutrophiles, CD8 lymphocytes, Tγδ lymphocytes, NKT cells, eosinophiles, basophiles or mastocytes, this list being in no way limitative. Advantageously, these cells are capable of reacting when the Fc region of an antibody binds to the Fc receptor expressed at their surface.

For the purposes of the invention, by “reaction of the cells expressing an Fc receptor” is meant any measurable cell reaction due to the interaction between the Fc receptor of these cells and the Fc region of the antibodies. This reaction can be intracellular or extracellular. By way of example, mention can be made of the measurement of one or more cytokines, measurement of the level of intracellular calcium, perforin, granzyme or nitrogen monoxide, this list being in no way limitative.

Advantageously, the aggregation is carried out using a F(ab′)₂ anti-IgG fragment. This means allows a particularly advantageous aggregation in terms of controlling the orientation of the Fc regions of the antibodies. In fact, each F(ab′)₂ fragment binds to two different antibodies to be tested, thus orienting the Fc regions of the antibodies to be tested in a suitable fashion. This orientation is particularly suited to interaction with the Fc receptors of the effector cells, and therefore to the activation of the effector cells.

The F(ab′)₂ anti-IgG fragments capable of being used in this embodiment can be directed against any fragment, part or domain of the antibodies, for example the Fc region or the Fab region or the whole of the IgG molecule. The F(Ab′)₂ fragments can also be of monoclonal or polyclonal origin.

In this embodiment of the invention, the concentration of antibodies to be tested and the F(ab′)₂ concentration can be controlled accurately. It is thus possible calculate the relationship between the two, making it possible to carry out reproducible tests regardless of the antibody preparation to be tested.

Particularly advantageously, this F(ab′)₂ anti-IgG fragment is an anti-Fab or an anti-F(ab′)₂ directed against the tested antibody preparation. This fragment can be a goat or rabbit fragment.

In this particular embodiment, each F(ab′)₂ fragment binds to the Fab parts of two antibody molecules to be tested, thus orienting the Fc regions of the antibodies to be tested in a suitable fashion to allow them to bind to the FcR and activate the cells expressing these FcRs at the surface.

According to another embodiment, the aggregation is an aggregation by heat. The antibodies are firstly heated so that they aggregate with each other (Step a) of the method of the invention), then placed in contact with the cells expressing an Fc receptor (Step b)). Any heating method suitable for aggregating the antibodies with each other can be used in the implementation of the method of the invention. By way of example, mention can be made of heating the antibodies at 60° C. for 30 minutes [1].

According to another embodiment of the invention, the aggregation is carried out by cross-linking the Fab regions to each other or the heavy and light chains to each other. For the purposes of the invention, by “cross-linking” is meant any bridging between two antibody molecules, such as to orient the Fc region of these antibodies homogeneously towards the CD16 receptors of the effector cells. Advantageously, an antibody can be involved in one or more bridgings. Thus, the antibodies are capable of forming a network, the orientation of which is suitable for optimal binding of the CD16 receptors carried by the effector cells. Advantageously, any means allowing cross-linking (i.e. bridging) of the antibodies with each other is suitable for implementation of the invention. By way of example, mention can be made of establishing chemical bonds, or bridging by UV radiation, this list being in no way limitative. Advantageously, the Fc receptor expressed by the effector cells is selected from CD16 (FcγRIIIa and FcγRIIIb), CD32 (FcγRIIa and FcγRIIb), and CD64. Particularly advantageously, CD16 is chosen.

In a preferred embodiment, the cells expressing the Fc receptor are cells transfected with the gene encoding said receptor. In this embodiment, it is possible to control the allotype(s) of the receptors presented by the effector cells. In fact, it is possible to carry out the method of the invention with cells having only one receptor chosen with regard to its properties, or a combination of these receptors, with regard to the desired test. For example, it is possible to implement the invention by choosing to use effector cells expressing only CD16, by transfecting cells with the gene coding for CD16. Thus, it is possible to dispense with another variability factor, namely the nature and the quantity of Fc receptor(s) present at the surface of the effector cells.

In a preferred embodiment of the invention, the cells expressing the Fc receptor are Jurkat cells expressing CD16, this line being cultured in the presence of a non-specific activator of these cells such as PMA. (Phorbol 12-Myristate 13-Acetate). One of the particularly advantageous aspects of implementation of the method according to the invention with this cell line is based on the fact that this line does not require activation prior to placing the effector cells in contact with the aggregated antibodies. In fact, certain effector lines need to be activated using one or more cytokine(s) for sufficiently significant expression of the Fc receptors (see for example document EP 1 298 219). This prior activation with cytokines is often random, resulting in both loss of time and difficulty in standardization of the experiments which is not easily overcome. Furthermore, the Jurkat line transfected with a expression vector encoding the CD16 receptor (“Jurkat CD16 line”) has the advantage of being immortalized and therefore of multiplying indefinitely in the culture media.

These cells have the advantage that they can be activated when they are doubly stimulated. In the method of the invention, activation of the Jurkat CD16 cells is carried out by PMA (which is an aspecific activator of T-cells) and binding of CD16 with the Fc region of an antibody. Activation of the Jurkat cells is shown by a release of IL-2 in the culture supernatant. As a result, the more functional the Fc region of an antibody vis-à-vis the CD16, the higher the quantity of IL-2 released in the supernatant.

Advantageously, measurement of the reaction of the cells resulting from the activation of the Fc receptors of said cells by the Fc regions of said antibodies is a measurement of the quantity of at least one cytokine produced by the cells expressing CD16 receptors. In fact, the activation of the effector cells is shown, amongst other things, by a release of IL-2 in the culture supernatant. For example, the concentration of cytokines in the culture medium can be measured by means of a commercially available ELISA (bioassay) test. Other methods can make it possible to assess the synthesis of a cytokine such as IL-2: these are the quantitative RT-PCT and Northern blot techniques for the assay of the messenger RNAs of IL-2 or Western blot and cytometry for quantification of intracellular IL-2 and IL-2 in the culture supernatant, this list being in no way limitative.

In an embodiment of the invention, measurement of the quantity of at least one cytokine is effected by carrying out an assay of the mRNAs of these cytokines, the quantity of these mRNAs being correlated with the level of expression of the corresponding cytokines, which shows the activation level of the effector cells.

Advantageously, at least one cytokine selected from IL-1, IL-2, IL-3, IL-4, IL-5, IL-6, MCP-1, TNFalpha (TNFα) and IFNgamma (IFNγ) is quantified, this list being in no way limitative.

Particularly advantageously, the measurement of the cell reaction is the quantification of interleukin IL-2.

The level of secreted interleukin IL-2 is correlated to an ADCC-type activity. In fact, there exists a strong correlation between the secretion of cytokines by the effector cells and the ADCC activity mediated by the CD16 of the effector cells (Document FR 02 11416). Thus, the method of the invention is particularly advantageous for selecting cytotoxic antibodies, in particular for a therapeutic use.

In another embodiment of the invention, measurement of the cell reaction is a measurement of the calcium influx, phosphorylation, transcription or apoptosis factors. The increase in these parameters is correlated to an activation of the effector cells, which shows the ability of the antibodies to activate the Fc receptors of the effector cells.

Advantageously, the method is suitable for evaluating the ability of a cell to produce a monoclonal antibody capable of interacting with the CD16 receptor, i.e. to assess the cytotoxicity of an antibody or an antibody preparation. In the literature, it has been demonstrated that the affinity of an antibody for CD16 is dependent on the characteristics of the Fc receptor such as for example the polymorphism of CD16 [2, 3], but also of the Fc region, or the fucose level of the oligosaccharide present on asparagine in position 297 of the heavy chains of the immunoglobulins plays a role in binding to the Fc receptors [4, 5].

The cell line producing antibodies can be any line capable of dividing, but is more particularly chosen from the CHO, YB2/0, SP2/0, SP2/0-AG14, IR983F, the Namalwa human myeloma, PERC6 cell lines, the CHO lines, in particular CHO-K-1, CHO-Lec10, CHO-Lec1, CHO-Lec13, CHO Pro-5, CHO dhfr-, Wil-2, Jurkat, Vero, Molt-4, COS-7, 293-HEK, BHK, K6H6, NS0, SP2/0-Ag 14 and P3X63Ag8.653, this list being in no way limitative.

Advantageously, the method is suitable for assessing the effectiveness and integrity of the Fc region of antibody preparations obtained after one or more purification stages.

Another application of the method of the invention is monitoring the stability of a preparation, in particular therapeutic, of monoclonal or polyclonal antibodies, placed under denaturing conditions.

The method of the invention is therefore particularly useful in monitoring preparations for therapeutic use over time. In fact, the Applicant monitored the activity of an antibody preparation stored under denaturing conditions over several months and showed that it decreases. Thus, a therapeutic preparation loses its activity over time when it is stored at a high temperature, for example at 40° C. Moreover, the method of the invention makes it possible to distinguish which part of the antibody is involved in this loss of activity, unlike the tests involving the cells carrying the target antigens. It is apparent that it is the Fc region of the antibodies which, once denatured, loses its affinity for CD16 (cf. Example 4).

Moreover, the method is advantageously suitable for measuring the functionality of the Fc region of an antibody. By “functionality of the Fc region of an antibody”, is meant for the purposes of the invention the ability of this Fc region to activate the effector cells via its binding to the Fc receptor, in particular to the CD16 receptor. The method of the invention being highly reproducible (cf example 2, paragraph 5), it can be used routinely for analyzing the functionality of antibody preparations, as the measurement efficiency is the same for antibodies of different sequence and/or specificity, as well as for screening highly cytotoxic antibodies.

Advantageously, the method is suitable for assessing the production of monoclonal antibodies by transgenic plants or transgenic mammals. By means of the implementation of the method according to the invention, the antibodies thus produced could be characterized with regard to the ability of their Fc region to activate an Fc receptor.

Advantageously, the method is suitable for selecting effective antibodies for a therapeutic treatment. By way of example, the antibodies are selected for which an increase greater than 100%, or 250%, advantageously 500% or preferably 1000% of the cytokine release level, for example of IL-2, is observed with respect to the control in which antibodies are absent or with respect to a given antibody as a negative reference.

Advantageously, the method is suitable for selecting antibody compositions the fucose content of which is less than 65%, and preferably less than 40%. In fact, it was demonstrated that the activity of antibody preparations was dependent on the fucose content on the glycannic unit in position 297 of the heavy chain.

The antibodies are constituted by heavy chains and light chains, linked together by disulphide bridges. Each chain is constituted, in the N-terminal position, by a specific variable region (or domain) of the antigen against which the antibody is directed, and in the C-terminal position by a constant region, constituted by a single CL domain for the light chains and several domains (CH₁, CH₂ and CH₃) for the heavy chains. The association of the variable domains and the CH₁ and CL domains of the heavy and light chains form the Fab parts of the antibody, which are connected to the Fc region by a very flexible hinge region, allowing each Fab to bind to the target antigen. The Fc region, mediator of the effector properties of the antibody, remains accessible to the effector molecules such as the FcγR receptors (FcgammaR). The Fc region, constituted by 2 globular domains CH₂ and CH₃, is glycosylated at the level of the CH2 domain with the presence, on each of the 2 chains, of a biantenna-type N-glycan, bound to the asparagine 297 (Asn 297).

An N-glycan of this type is presented in the following general form (presented form (“G0”, to which other sugars can be added):

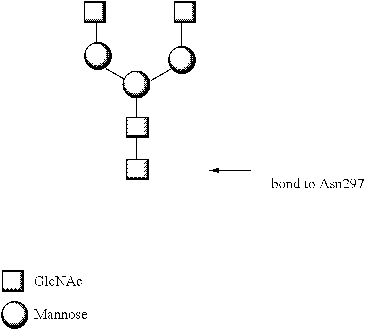

Thus, in the antibody composition according to the invention, by “fucose” is meant the fucose carried by these N-oligosaccharides. The fucose molecule, when it is present, is bound to the N-acetylglucosamine (GlcNAc) of the N-oligosaccharide, this GlcNAc being itself bound to the Asn 297. Each of the 2 N-glycans carried by each of the 2 heavy chains of each antibody, may or may not carry a fucose molecule. Thus, each antibody can comprise 0, 1 or 2 fucose molecules respectively, according to whether none of its N-glycans carries fucose, only one of its N-glycans carries a fucose molecule, or both its N-glycans each carry a fucose molecule. Thus, by “antibody composition the fucose content of which is less than 65%” is meant an antibody composition, of which, from the totality of the glycannic structures carried by each glycosylation site (Asn 297) of the antibodies of the composition, less than 65% comprises a fucose molecule.

It has been demonstrated by the Applicant that such compositions have a particularly advantageous ADCC activity.

Preferably, compositions of antibodies are selected, the fucose content of which is comprised between 20% and 45%, or between 25% and 40%. By means of the method of the invention, a quantitative relationship has been shown between the functional activity and the fucose level in the sense of an increase in the functional activity of the antibodies in relation to the CD16 when the fucose level reduces in the antibody composition (preparation).

Another object of the invention relates to a method for the preparation of a monoclonal antibody composition comprising the following steps:

a) obtaining antibodies from a hybridoma, a heterohybridoma, or any animal, plant or human transfected cell line using one or more vectors to express said antibody,

b) aggregating the antibodies obtained in step b) by a F(ab′)₂ anti-IgG fragment,

c) adding the antibodies obtained in step b)

in a reaction mixture comprising: effector cells comprising cells expressing CD16,

Phorbol 12-Myristate 13-Acetate (PMA)

d) measuring the quantity of at least one cytokine produced by the cell expressing CD16,

e) selecting the antibody composition(s) for which an increase greater than 0.5, 1, 2, 5, 10, 100 or 500 times the measured quantity of cytokine is measured with respect to the control in the absence of antibodies or in the presence of a given antibody as a negative reference.

Advantageously, the effector cells comprising cells expressing CD16 are Jurkat CD16 cells.

In an embodiment of the invention, the measurement of the quantity of at least one cytokine is effected by carrying out an assay of the mRNAs of these cytokines, the quantity of these mRNAs being correlated to the level of expression of the corresponding cytokines, which shows the level of activation of the effector cells.

For example, at least one cytokine selected from IL-1, IL-2, IL-3, IL-4, IL-5, IL-6, MCP-1, TNFalpha (TNFα) and IFNgamma (IFNγ) is quantified, this list being in no way limitative.

Advantageously, the measurement of the cell reaction is the quantification of interleukin IL-2.

The level of secreted interleukins, for example IL-2, is correlated to an ADCC-type activity. In fact, a strong correlation exists between the secretion of cytokines by the effector cells and the ADCC activity mediated by the CD16 of the effector cells (Document FR 02 11416).

In another embodiment of the invention, the measurement of the cell reaction is a measurement of the calcium influx, phosphorylation, transcription factors or apoptosis. The increase of these parameters is correlated to an activation of the effector cells, which shows the ability of the antibodies to activate the Fc receptors of the effector cells.

Advantageously, the measurement of the quantity of at least one cytokine produced by the cell expressing CD16 is a measurement of the quantity of IL-2 produced by the cell expressing CD16.

Another subject of the invention relates to a kit for the implementation of a biological test for measuring the activity of therapeutic antibodies comprising the elements necessary for the implementation of one of the methods previously described, and comprising in particular (i) means for aggregating said therapeutic antibodies, (ii) cells capable of expressing an Fc receptor, (iii) means for measuring the reaction of said cells capable of expressing an Fc receptor when the Fc receptor of said cells is activated by the Fc region of said antibodies, and (iv) the other elements necessary for the implementation of the method according to any one of the preceding claims.

Kit for the implementation of a biological test for measuring the activity of therapeutic antibodies comprising (i) means for aggregating said therapeutic antibodies, (ii) cells capable of expressing an Fc receptor, (iii) means for measuring the reaction of said cells capable of expressing an Fc receptor when the Fc receptor of said cells is activated by the Fc region of said antibodies, and (iv) the other elements necessary for the implementation of the method according to any one of the preceding claims.

In an advantageous embodiment of the invention, the means for measuring the reaction of said cells included in said kit are means for quantifying at least one cytokine produced by said cells.

In an advantageous embodiment of the invention, said means for quantifying at least one cytokine of said kit are means for assay of the mRNAs of said cytokines.

In another embodiment of the invention, the means for measurement of the reaction of said cells in said kit are means for measuring the calcium influx, phosphorylation, transcription factors or apoptosis.

Other aspects and advantages of the invention are described in the examples which follow, which must be considered as illustrative and do not limit the scope of the invention.

### DESCRIPTION OF THE FIGURES

FIG. 1: (A) Calibration curve defined by the measurement of the average intensities of fluorescence of the 5 populations of beads, (B) CD16 expression profile of the JCD16⁺ Val population obtained by FACS analysis of an anti-CD16 labelling.

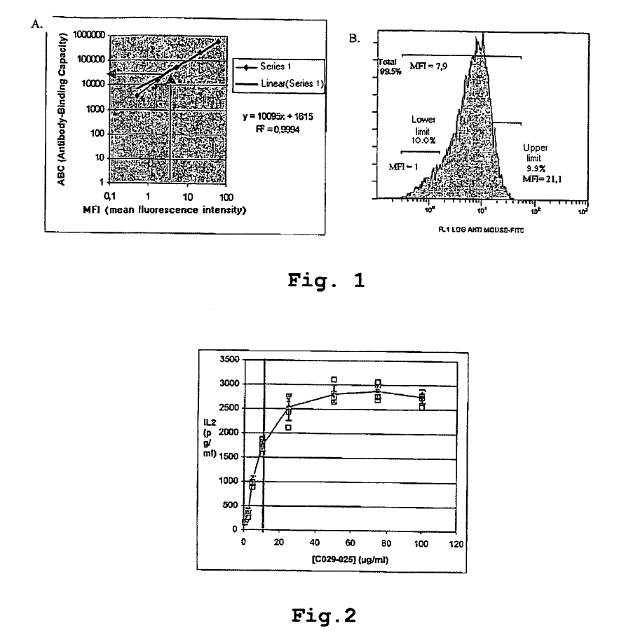

FIG. 2: Dose-response curves of an aggregated anti-D antibody (batch C029-025). Curve specific to JCD16+ Phe cells. The red vertical line delimits the range of concentrations (0.625 to 10 μg/ml) used for the remainder of the tests.

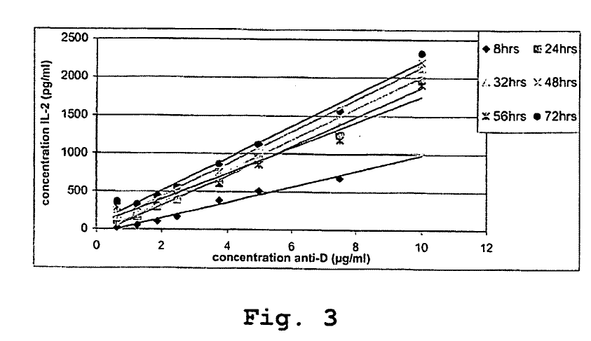

FIG. 3: kinetic diagram of IL-2 secretion for the JCD16⁺ Phe cells.

FIG. 4: Influence of the fucose level of the antibodies on their activity. (A) test of activity of the anti-Gp120 antibodies VIH (100% fucose), AD1 (100% fucose), T125 CHO (81% fucose), R270 (64% fucose), R297 (40% fucose) and R297 (25% fucose) in the test in the presence of target cells, (B) test of activity of the anti-Gp120 HIV antibodies (100% fucose), AD1 (100% fucose), T125 CHO (81% fucose), R270 (64% fucose), R297 (40% fucose), C029-025 (33% fucose) and R297 (25% fucose) in the test without target cells.

FIG. 5: Monitoring of the stability of batch 05-081 at 40° C. by monthly measurement of its activity.

FIG. 6: Effect of temperature on the activity of an anti-D monoclonal antibody vis-à-vis its ability to activate CD16 at 10 and 5 μg/ml, the measurements being carried out by ELISA test.

FIG. 7: Effect of temperature on the activity of an anti-D monoclonal antibody vis-à-vis its ability to activate CD16 at 10 and 5 μg/ml, the measurements being performed by quantitative RT-PCR test.

### EXAMPLES

### 1. Cell Culture

1.1 Cell Lines

The Jurkat line, clone E6-1 (No. ATCC TIB-152), was deposited at the ATCC by A. Weiss [6] in 1984. The clone E6-1 was obtained from the wild-type Jurkat line. This was isolated in 1977 by Schneider et al. [7] from the peripheral blood of a child aged 14 years with acute lymphoblastic leukaemia. This line, expressing the T cell markers is capable of producing interleukin 2 (IL-2) when it is stimulated by two different activation signals. However, the level of IL-2 secreted varies according to the origin of the line and the handling conditions. This is why Jurkat clones of different origins have been transfected so that they express the FcγRIIIa receptor at membrane level. A line was obtained by transfection by an expression vector encoding a human chimeric FcγRIIIa (extracellular domain of CD16 associated with the transmembrane and intracellular domains of the γ chain) and the neomycin selection gene. These cells will be called JCD16⁺ Phe as they express the human CD16 having a phenylalanine in position 158.

1.2 Culture and Subculture Media

The JCD16⁺ Phe cells are kept in IMDM medium (Iscove's Modified Dulbecco's Medium)+4 mM L-Glutamine+25 mM HEPES buffer (Gibco, Invitrogen) with the addition of 10% FCS (Foetal Calf Serum) irradiated with γ-rays and decomplemented (Invitrogen), and a neomycin analogue (G418 sulphate) at 0.5 mg/ml (Promega). This cell line is maintained in culture at the rate of two subcultures per week with a seeding rate of 0.2.10⁶ cells/ml.

### 2. Flow Cytometry

The different labellings are analyzed on an EPICS XL cytometer (Beckman Coulter).

2.1 Phenotyping of the JCD16⁺ Phe Cell Line

This phenotyping relates to the analysis of different membrane markers which are CD45 (marker of the leucocyte cells), CD19 (specific marker of the B lymphocytes), CD2, CD3, CD4 and CD8 (specific markers of T-lymphocytes), CD58 (ligand of CD2), and also the different chains of the IL-2 receptor (CD25 or IL-2 Rα, CD122 or IL-2 Rβ and CD132 or IL-2 Rγ). The labellings are carried out on 10⁶ cells in 100 μl of PBS 1× (phosphate buffer). The conditions are the following:

10 μl of anti-CD2 FITC (2-fluorescein isothio-cyanate, Coulter Clone, Beckman Coulter)+10 μl of anti-CD3 RD1 (R-phycoerythrin, Coulter Clone, Beckman Coulter)+10 μl of anti-CD19 ECD (R-phycoerythrin/Texas Red tandem dye, Coulter Clone, Beckman Coulter)+10 μl of anti-CD16 PC5 (R-phycoerythrin/cyanine 5 tandem dye, IOTest, Beckman Coulter).

10 μl of anti-CD45 FITC (Coulter Clone, Beckman Coulter)+10 μl of anti-CD3 RD1 (Coulter Clone, Beckman Coulter)+10 μl of anti-CD4 ECD (Coulter Clone, Beckman Coulter)+10 μl of anti-CD8 PC5 (Coulter Clone, Beckman Coulter).
10 μl of anti-CD58 PC5 (IOTest, Coulter Clone).
5 μl of anti-CD122 FITC (Coulter Clone, Beckman Coulter)+10 μl of anti-CD132 PE (Phycoerythrin, BD Pharmigen)+10 μl of anti-CD25 PC5 (IOTest, Coulter Clone).

In parallel with these four labellings, isotypical controls are carried out and correspond respectively to:
10 μl of IgG1-FITC (Coulter Clone, Beckman Coulter)+10 μl of IgG1-RD1 (Coulter Clone, Beckman Coulter)+10 μl of IgG2b-ECD (Coulter Clone, Beckman Coulter)+10 μl of IgG1-PC5 (IOTest, Beckman Coulter).
10 μl of IgG1-FITC (Coulter Clone, Beckman Coulter)+10 μl of IgG1-RD1 (Coulter Clone, Beckman Coulter)+10 μl of IgG1-CD4 ECD (Coulter Clone, Beckman Coulter)+10 μl of IgG1-PC5 (IOTest, Beckman Coulter).
10 μl of IgG2a-PC5 (IOTest, Coulter Clone).
5 μl of IgG1-FITC (Coulter Clone, Beckman Coulter)+10 μl of IgG1-PE (BD Pharmigen)+10 μl of IgG2a-PC5 (IOTest, Coulter Clone).

The cells are incubated in the presence of the different monoclonal antibodies for 30 minutes in the dark, at ambient temperature. After washing the cells in PBS 1× and centrifugation at 1200 rpm for 5 minutes, the labellings are directly analyzed by cytometry.

2.2 Determination of the Number of CD16 Sites

The number of FcγRIIIa receptors expressed at the cell surface is assessed according to the QIFIKIT (Dako Cytomation) technique. This technique requires prior determination of the saturating dose of anti-CD16 antibodies (clone 3G8, anti-CD16 IgG1 conjugated with fluorescein, Immunotech) necessary to occupy all the antigenic sites at the surface of the cells. The cells (0.25.10⁶/100 μl of physiological saline) are incubated with increasing doses of anti-CD16 3G8 for 30 minutes at ambient temperature. After a washing in physiological saline followed by centrifugation at 1200 rpm for 5 minutes, the cells are then incubated with 50 μl of PE labelled anti-mouse IgG F(ab′)₂ (H+L) diluted to 1/20^{th} (Beckman Coulter) for 30 minutes in the dark. At the end of the incubation, the cells are washed and directly analyzed by FACS (fluorescence-activated cell sorting).

The determination of the number of CD16 sites is carried out on 2.5.10⁵ cells according to the protocol supplied in the kit (No. K0078). The cells are incubated with 500 ng antibodies of interest for 30 minutes at ambient temperature. After a washing and centrifugation (5 minutes at 1200 rpm), the cells are incubated with 100 μl of FITC-coupled anti-mouse IgG F(ab′)₂ diluted to 1/50^{th} (Qifikit), for 30 minutes, at ambient temperature and in the dark. The cells are then washed and analyzed directly by FACS.

### 3. Test of Production of IL-2 by the Jurkat Cells

3.1 Test in the Presence of Erythrocytes Carrying the Specific Antigen

3.1.1 Preparation of the Samples for Testing

Samples of anti-D antibodies are tested according to an eight-point scale of concentrations which are: 3.125 ng/ml, 6.25 ng/ml, 9.4 ng/ml, 12.5 ng/ml, 18.75 ng/ml, 25 ng/ml, 37.5 ng/ml and 50 ng/ml. Each concentration is obtained by dilution of the sample in IMDM+5% decomplemented FCS.

3.1.2 Preparation of the Target Cells

The target cells are Rhesus D⁺ erythrocytes obtained from donors, preferably O⁺. They are treated with papain (Bio-Rad). This is added volume/volume to the globular pellet and left to incubate for 10 minutes at 37° C. The reaction is stopped by the addition of a large volume of physiological saline (NaCl 0.9%, Ecotainer, B BRAUN). The cells are then washed three times in 0.9% NaCl and centrifuged at 3000 rpm for 5 minutes for the two first washings and 10 minutes for the final washing. The globular pellet is then diluted in IMDM+5% FCS in order to obtain a cell suspension at a concentration of 8.10⁶ cells/ml (0.08%).

3.1.3 Preparation of the PMA Solution

A PMA solution (10 μg/ml, Sigma) at a concentration of 40 ng/ml is prepared by dilution to 1/250^{th} in IMDM+5% FCS.

3.1.4 Preparation of the Effector Cells

The Jurkat cells, subcultured between 48 and 72 hours before the test, are counted on Malassez slides in order to define the volume of cell suspension to be sampled to have available 10⁷ cells (quantity necessary for one micro-plate). The volume of cell suspension is centrifuged for 10 minutes at 1200 rpm. The cell pellet obtained is then re-suspended in IMDM+5% FCS at a concentration of 2.10⁶ cells/ml. A new count is carried out in order to verify the concentration of the suspension and if necessary it is adjusted accurately by adding IMDM+5% FCS.

3.1.5 Carrying Out the Test

In a plate with 96 U-shaped wells, the following is placed in each well:

50 μl of anti-D antibodies to be tested,

50 μl of the globular suspension (i.e. 4.10⁵ erythrocytes per 50 μl),

50 μl of the cell suspension (i.e. 10⁵ cells per 50 μl),

50 μl of PMA (i.e. 2 ng per 50 μl).

For each plate, an anti-D reference solution is deposited as well as a control sample corresponding to an anti-D polyclonal antibody (Rhophylac 300 TM, Biotest). The wells are homogenized by stirring. The plate is incubated overnight at 37° C. The following day, the cells are decanted by centrifugation for 1 minute at 125 g. The supernatant is removed and the IL-2 concentration is determined by ELISA assay.

3.2 Test in the Absence of Antigen Target

The antibodies studied are tested as previously according to a scale of eight concentrations defined in section 3.1.1. In this test, the erythrocytes are substituted by a specific F(ab′)₂ anti-IgG fragment (1.3 mg/ml, Jackson Immuno-Research Laboratories). This is also diluted in IMDM+5% FCS at eight different concentrations. The anti-D and the F(ab′)₂ anti-IgG antibodies are studied in the ratio of 1/1.5. The PMA solution is prepared at a concentration of 10 ng/ml. The Jurkat cells are diluted in IMDM+5% FCS at a concentration defined in section 2.1.4.

In a plate with 96 U-shaped wells, the following is placed in each well:

50 μl of anti-D antibodies to be tested,

50 μl of specific F(ab′)₂ anti-IgG fragment,

50 μl of cell suspension,

50 μl of PMA (i.e. 0.5 ng per 50 μl).

The wells are homogenized by stirring. The plate is incubated overnight at 37° C. The following day, the cells are settled by centrifugation for 1 minute at 125 g. The supernatent is removed and the IL-2 concentration is determined by ELISA assay or by the assay of messenger RNAs (mRNAs) coding for IL-2.

3.3 Dosage of the IL-2 of the Cell Supernatent by the ELISA Technique

3.3.1 Materials and Methods

The quantity of IL-2 secreted is assayed according to the operating method of the Duoset® human IL-2 kit (DY202, R&D Systems). A capture antibody is distributed into each well of a micro-plate. After saturation with a bovine albumin solution, the supernatants to be assayed are applied at different dilutions, as well as a reference scale of IL-2. A biotinylated human anti-IL-2 antibody is then added, followed by a solution of streptavidine peroxydase HRP. After addition of the substrate (Tetramethylbenzidine), a blue colouration develops. After the reaction is stopped with sulphuric acid (H₂SO₄), the optical density of each well is determined by reading the plate at 450 nm. The IL-2 concentration for each well is calculated by means of the Biolise software which determines the regression curve of the IL-2 scale ([IL-2]=a [antibodies]²+b [antibody]+c) and which takes account of the dilution factor of the sample in each well.

3.3.2 Interpretation of the Results

The IL-2 concentrations determined by ELISA assay make it possible to determine the activity of each anti-D antibody tested. This is calculated in the following manner:

Starting from a reference antibody, a curve of the second-degree equation is established by plotting the IL-2 concentrations measured as a function of the antibody concentration range. For each sample concentration applied, the equivalent reference anti-D concentration is calculated by means of the second-degree equation of the curve. Each of the equivalent anti-D values is brought to the theoretical concentration of 50 ng/ml for the test with target cells or 10 μg/ml for the test without target cells, taking account of the dilution of the antibody studied. The average value for each sample is then calculated. To determine the activity percentage of the sample, an activity of 100% is arbitrarily attributed to the reference. It is then only necessary to calculate the following relationship:

average of the recalculated concentrations of the sample/average of the recalculated concentrations of the reference.

Thus, if the percentage of activity is less than 100 for an unknown anti-D, this means that the activity of this antibody is lower than that of the reference antibody. On the other hand, if the percentage of activity is above 100, this means that the anti-D studied has an activity greater than the reference.

3.4 Assay of the IL-2 by Assay of the mRNAs Coding for IL-2

The technique rests on the assay of the messenger RNAs (mRNAs) coding for IL-2. Firstly, the total RNA is extracted from the cells. The RNA is converted to complementary DNA (cDNA) using an enzyme called reverse transcriptase. The genetic sequence corresponding to IL-2 is detected by means of specific primers by the quantitative Polymerase Chain Reaction (PCR) technique. In order to compare the samples to each other, it is necessary to standardize the assay. This is carried out by simultaneous assay of the mRNAs coding for a ubiquitous or constitutive gene, the expression level of which is identical in all the cells regardless of the culture and stimulation conditions. The ubiquitous gene chosen in these tests is b-actin, which is one of the constituents of the cytoskeleton of the cells. The more activated the cell, the higher will be the IL-2 cDNA/b-actin cDNA ratio. The mRNAs coding for IL-2 are transitory. Thus the time is determined for which the gene expression is maximum after stimulation under the experimental conditions (4-6 hours).

3.4.1 Materials and Methods

1) Cell Culture

The test is carried out under the following conditions:
200 μl of JURKAT CD16+ cell suspension at 10⁷ cells /ml;
Anti-D monoclonal antibody mAb with F(ab′)₂:
200 μl of anti-D mAb at 10 μg/ml with 200 μl of F(ab′)₂ (Jackson Immuno-Research) specific anti-fragment at 15 μg/ml; or
200 μl of anti-D mAb at 5 μg/ml with 200 μl of F(ab′)₂ (Jackson Immuno-Research) specific anti-fragment at 7.5 μg/ml;
200 μl of PMA at 40 ng/ml.

All the solutions used are prepared in IMDM medium with 5% FCS.

The cells are then incubated at 37° C. in an incubator under a controlled atmosphere (95% air+5% CO₂).

2) Extraction of the Total RNA

The total RNA is extracted using 2 commercial kits:

(QIAGEN):
QIAshredder kit;
RNeasy Protect mini kit;

then the extracted RNA is assayed by spectrophotometry at 260 nm.

3) Reverse Transcriptase Step

The cDNA is obtained by incubating for 1 hour at 42° C.:
1 μg of linearized RNA (heating to 65° C. followed by immediate cooling to 4° C.);
oligo dT primer;
dATP, dCTP, dGTP and dTTP;
Dithiothreitol (DTT);
RNAase inhibitor;
Reverse transcriptase

4) PCR Step

The PCR step is carried out using the specific primers described below:

For IL-2
IL-2 H1: AAC AGT GCA CCT ACT TCA AG
IL-2 H2: GTT GAG ATG ATG CTT TGA CA
For b-actin
BACT H1: GGG TCA GAA GGA TTC CTA TG
BACT H2: GGT CTC AAA CAT GAT CTG GG

The quantitative PCR is carried out using the Applied Biosystems 7300 apparatus and the use of the Power Syber Green master mix kit (Applied Biosystems). The programme used for the 2 primer pairs is
1 dehybridization cycle at 95° C. for 10 minutes;
40 cycles:
Dehybridization at 95° C. for 10 seconds;
“Annealing” (primer coupling) at 60° C. for 15 seconds;
Elongation at 72° C. for 60 seconds.

At the end of the PCR, the amplified products are denatured slowly and progressively to define the dissociation curve and the Tm (melting temperature) in ° C. which are specific to the amplified product.

3.4.2 Results

Two anti-D mAb preparations at 2 different concentrations (5 and 10 μg/ml) were compared. One of the preparations was stored at 5° C. while the other was stored for 3 months at 40° C.

For the quantitative RT-PCR technique, the samples were taken 4 hours after the start of the cell test culture to extract the RNA.

For the technique of assay of IL-2 in the culture supernatant by ELISA, the samples were taken 16 hours after the start of culture.

1) ELISA Results

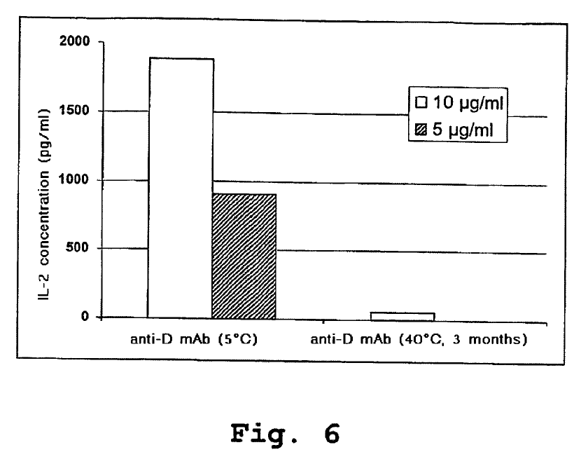

The IL-2 concentration present in the supernatants 16 hours after the setting up of the test was quantified using an assay kit (R&D Systems). The results obtained are shown in FIG. 6.

2) Quantitative RT-PCR Results

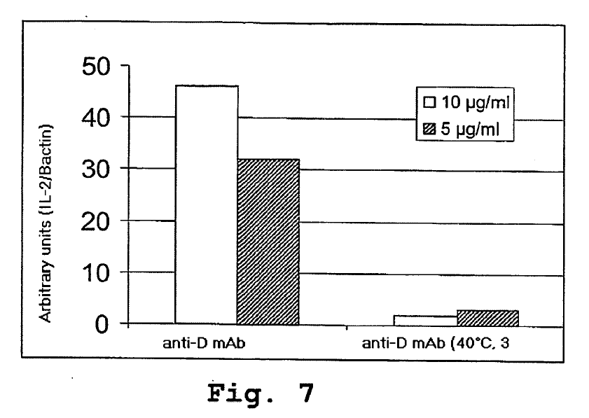

Expression of the IL-2 gene in the Jurkat CD16+ cells, 4 hours after the set-up of the test was estimated by RT-PCR in real time. The results have been standardized in relation to the b-actin ubiquitous gene. The results are shown in FIG. 7.

3.4.3 Conclusion

There is a correlation between the direct assay method of the cytokine IL-2 concentration in the supernatant of the stimulated Jurkat CD16+ cells and the assay method of the expression of its gene by quantitative RT-PCR.

As for the ELISA test, the intensity of expression of the IL-2 gene depends on the nature of the mAb studied and its concentration. This technique can therefore be used to study the interactions between the Fc region of an mAb and the CD16 receptor.

### Example 1

### 1. Characterization of the Cell Lines

1.1 Genotyping of the Lines

Prior to the development of this test, the genotyping of each line was carried out by allelic discrimination by Q-PCR (Applied 7300). The genotyping was verified by RT-PCR.

| | DNA | RNA |
|---|---|---|
| Jurkat CD16⁻ | T/G | ND |
| Jurkat CD16⁺ Phe | ND | T i.e. Phe |

Analysis of the genotype of the lines by Q-PCR (DNA) and RT-PCR(RNA).

The Jurkat CD16⁺ line does not express the FcγRIII receptor at its membrane surface. The Jurkat CD16⁺ line called Phe does express the T (Phe) genotype

1.2 Phenotyping of the Lines

Analysis of the membrane markers by FACS marking made it possible to obtain the following results:

| | Jurkat CD16⁺ Phe |
|---|---|
| CD45 (B220) | ++ |
| CD19 Pan B (B4) | − |
| CD2 Pan T (LFA3-R) | ++ |
| CD3 Pan T | ++ |
| CD4 | ++ |
| CD8 | − |
| CD 16 FcγRIII | ++ |
| CD58 (LFA3) | + |
| CD25 (IL-2 Rα) | − |
| CD 122 (IL-2 Rβ) | − |
| CD132 (IL-2 Rγ) | + |

Analysis by cytometry of the membrane markers of the two transfected Jurkat lines.

The lines therefore have the following phenotype CD45⁺, CD2⁺, CD3⁺, CD4⁺ CD16⁺, CD58⁺ and CD132⁺.

1.3 Determination of the Number of CD16 Sites

The purpose of this experiment is to quantify the number of CD16 antigen sites by an indirect marking analyzed by cytometry. The technique is based on the use of five populations of beads of 10 μm diameter coated with monoclonal antibodies (mouse anti-human CD5) in defined increasing quantities (10³ to 10⁶ Antibody-Binding Capacity or ABC). These different populations allow the construction of a calibration line corresponding to the mean fluorescence intensities (MFI) against the antibody binding capacity (ABC). The cells are saturated with the primary antibodies of interest (anti-CD16) which is revealed by a labelled secondary antibody, also introduced under saturating conditions. While respecting these conditions, the number of bound primary antibodies corresponds to the number of antigen sites present at the surface of the cells. As a result, the fluorescence is correlated to the number of primary antibodies bound to the cells. The ABC of the cells is determined by means of the calibration line.

The table below is the summary table of the determination of the number of CD16 sites for the line studied (study carried out on several subcultures):

| | P12 | P13 | P16 | P23 | P23 |
|---|---|---|---|---|---|
| Jurkat | (0.5 mg/ml | (0.5 mg/ml | (0.5 mg/ml | (0.5 mg/ml | (1 mg/ml |
| CD16⁺Phe | G418) | G418) | G418) | G418) | G418) |
| Total | 54000 | 68000 | 68000 | 89000 | 121000 |
| lower 10% | 16000 | 19000 | 19000 | 27000 | 36000 |
| upper 10% | 121000 | 160000 | 163000 | 187000 | 250000 |

After thawing a vial obtained from a library, each line is monitored over several subcultures in a selective medium in order to verify that the CD16 expression is maintained.

Thus, it was noted that after several passages, the CD16 expression was maintained and varied only very little under identical culture conditions from one subculture to another. Moreover, the pressure of selection and the results tend to show that a selection of clones with a high expression potential is operating. Furthermore, this technique makes it possible to analyze accurately the heterogeneity of the CD16 expression at the surface of the cells, by determination of the number of sites for extreme populations (the lower 10% and the upper 10%).

### Example 2

### CD16 Activation Test

1 Specificity of the Test

The specificity of the test was verified as follows: several controls are introduced into the test of secretion of IL-2 by the Jurkat cells in order to confirm the cell activation mechanism. A detection threshold limit of 15 pg/ml is defined by the last point of the IL-2 reference scale supplied in the assay kit. Below this threshold, the secretion of IL-2 by the cells is considered as undetectable. The table below is the synoptic table of the average IL-2 levels over n experiments and the standard deviations of each control introduced into the IL-2 production test. (<LT=below the limit threshold of 15 pg/ml)

| | | JCD16+ |
|---|---|---|
| | | Phe cells |
| Cells only | No. of experiments | n = 8 |
| | Average | <LT |
| F(ab′)₂ + PMA | No. of experiments | n = 8 |
| | Average | 52 |
| | Standard deviation | 48 |
| Anti-D + PMA | No. of experiments | n = 9 |
| | Average | 277 |
| | Standard deviation | 124 |
| Anti-D + F(ab′)₂ | No. of experiments | n = 8 |
| | Average | <LT |
| | Standard deviation | |
| Anti-D + F(ab′)₂ + | No. of experiments | n = 9 |
| PMA | Average | 1347 |
| | Standard deviation | 772 |
| PMA + Ionomycin | No. of experiments | n = 8 |
| | Average | 7970 |
| | Standard deviation | 2736 |
| PMA only | No. of experiments | n = 8 |
| | Average | 66 |
| | Standard deviation | 61 |
| Ionomycin only | No. of experiments | n = 8 |
| | Average | <LT |
| | Standard deviation | |

The assay of the culture supernatant of the cells alone makes it possible to define their basal secretion level which is thus found to be undetectable. This control makes it possible to verify that in the absence of any stimulus, the cells do not secrete IL-2. Moreover, these controls make it possible to confirm the need for two stimuli (PMA and anti-D/F(ab′)₂ complex) to result in activation of the cells. In fact, the secretion of IL-2 is minimal, or even zero when one of the two stimuli is absent (controls PMA+F(ab′)₂ or F(ab′)₂+anti-D). Similarly, activation of the CD16 requires the aggregation of the antibodies as, in the absence of F(ab′)₂, a secretion of IL-2 is obtained but in a manner greatly inferior to a secretion obtained in the presence of PMA and anti-D/F(ab′)₂ aggregates. Moreover, during each experiment, the maximum activation is verified by the use of PMA and ionomycin. These results therefore make it possible to demonstrate the need for a double stimulation of the JCD16⁺ cells in order to obtain an IL-2 secretion.

2. Dose Effect and Determination of the Antibody Concentration Range

In order to determine the optimum range of anti-D concentrations for the remainder of the experiments, a dose-response test (represented by the following two curves) was carried out. To this end, a range of concentrations from 1 to 100 μg/ml of R297 antibodies (batch C029-025) was tested in the reaction medium. The experiment was carried out in quintuplicate (see Figure No. 2).

For low anti-D concentrations, the curve of the IL-2 concentration in the supernatant after 24 hours of stimulation is proportional to the anti-D concentration introduced into the test. For high anti-D concentrations, the curve reaches a plateau.

3. Kinetic Activation and Determination of the Incubation Time

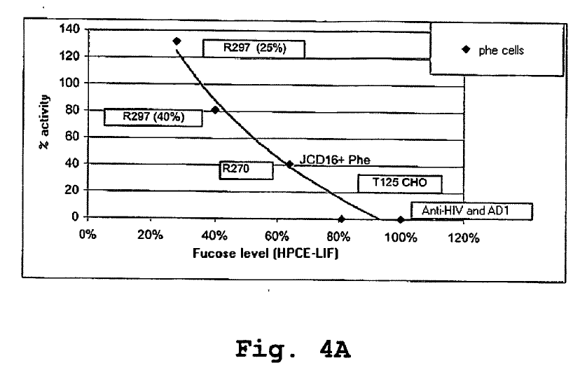

In order to determine the optimum duration of incubation, a kinetic study of the IL-2 secretion of the two cell lines was carried out. To this end, the cells were stimulated for 8, 24, 32, 48, 56 or 72 hours by a defined range of anti-D concentrations (from 0.625 to 10 μg/ml of batch C029-025). The IL-2 concentrations in the supernatant were assayed for each anti-D concentration, and for each duration of incubation, thus making it possible to obtain the straight lines in FIG. 3.

This study makes it possible to show that after stimulation for 8 hours, the cells secrete only very little IL-2. From 24 hours, the IL-2 levels reach the maximum values. After stimulation for 24 hours, the curves even tend to become superimposed. The optimum duration of incubation was fixed at 24 hours in order to limit the duration of the test.

4. Effect of the Cell Concentration in the Test

It is relatively difficult to standardize the cell concentration in a biological test. The purpose of this study is to verify whether or not the percentage activity of a given sample is dependent on the cell concentration. To this end, during three independent experiments, a sample of anti-D monoclonal antibodies (batch R297No. 05-081, T₀) was tested with five different cell concentrations (C₁=10⁵ cells/well, C2=2.5.10⁵ cells/well, C3=5.10⁵ cells/well, C4=7.5.10⁵ cells/well and C5=10⁶ cells/well). Its percentage activity was then determined by comparison with a reference anti-D (batch C029-025) the percentage activity of which is set arbitrarily at 100%. The results obtained for each concentration during the three experiments, are presented in the following tables.

| | JCD16⁺ Phe cells | | | | | |
|---|---|---|---|---|---|---|
| | Experiment 1 | | Experiment 2 | | Experiment 3 | |
| | % Activity | | % Activity | | % Activity | |
| concentrations | Sample/Ref | CV | Sample/Ref | CV | Sample/Ref | CV |
| C1 | no result obtained | | 143 | 7 | 135 | 11 |
| C2 | 127 | 12 | 131 | 12 | 116 | 11 |
| C3 | 88 | 5 | 127 | 9 | 138 | 9 |
| C4 | 131 | 8 | 121 | 7 | 119 | 10 |
| C5 | 120 | 3 | 123 | 6 | 115 | 9 |
| Average | 117 | 7 | 129 | 8 | 125 | 10 |
| Standard | 20 | 4 | 9 | 2 | 11 | 1 |
| deviation | | | | | | |
| CV | 17 | | 7 | | 9 | |

Percentage Activity of the Sample R297No. 05-081 Tested in Three Independent Experiments Involving Five Cell Concentration Conditions, for Jurkat CD16⁺ Phe Cells.

In this study, analysis of the coefficients of variation (CV) shows that the three experiments give a comparable activity of the sample No. 05-081 regardless of the cell concentration studied.

5. Reproducibility of the Test

A biological variability is introduced when cells are used in a test, even in the case of cell lines. Thus, with the aim of standardizing a test, it is necessary to ensure that the latter is well reproducible. This is even more necessary when the test is to be implemented in order to assess the influence of the purification method on the activity of an antibody, but also when stability studies are carried out over time with therapeutic preparations.

The activity of a sample (batch C029-025) was thus tested in several experiments. Its percentage activity is calculated in relation to a reference which corresponds to the same sample, the activity of which is set arbitrarily at 100%.

JCD16+ Phe

C029-025 (0.727 g/L)

| | IL-2 level at 10 μg/ml | % Sample/Ref | |
|---|---|---|---|
| Experiment | of anti-D | Activity | CV |
| No. 1 | 1276 | 92 | 7 |
| No. 2 | 2022 | 107 | 5 |
| | 2182 | 106 | 6 |
| No. 3 | 2210 | 104 | 10 |
| | 2096 | 103 | 8 |
| No. 4 | 1795 | 96 | 17 |
| | 1975 | 101 | 13 |
| | 2078 | 96 | 12 |
| No. 5 | 1704 | 105 | 11 |
| | 1160 | 96 | 8 |
| | 1309 | 98 | 4 |
| No. 6 | 419 | 105 | 5 |
| | 313 | 96 | 10 |
| Average | 1580 | 100 | 9 |
| Standard | 644 | 5 | 4 |
| deviation | | | |
| CV | 41 | 5 | |

Reproducibility Test: Determination of the Percentage Activity of a Sample (Batch C029-025) During n Experiments.

In the case of a biological test, a maximum coefficient of variation of 15-20% is accepted for the test to be valid. In the case of our test, the CVs obtained (5% for the JCD16₊ Phe cells) are therefore below the acceptance level. The use of this test as a routine is therefore possible for monitoring the stability of samples but also during the verification of the level of activity of a sample during its purification method.

### Example 3

### Evaluation of the Cytotoxicity of an Antibody vis-à-vis CD16

It has been demonstrated in the literature that the fucosylation level of the oligosaccharides present at the level of the heavy chains of the immunoglobulins influences the effector properties of the antibodies. In fact, a low fucose level would increase the antibody's ability to bind to CD16 [26]. Moreover, this mechanism would be totally independent of the polymorphism of the CD16 [27]. In this study, we are therefore interested in the impact of the fucose level on the functional activity of the antibodies. LFB has various monoclonal antibody preparations which have been characterized by their fucose level present on the glycan chains. A 100% fucosylated antibody corresponds to an immunoglobulin, the two glycan chains of which, in position 297 of the heavy chain, are totally fucosylated. Different samples having various fucose levels were studied in the test in the presence of target cells and in the test without target cells:
an anti-HIV Gp120 with 100% fucose,
AD1 (anti-D) with 100% fucose,
T125 CHO (anti-D produced in CHO cells) with 81% fucose,
R270 (anti-D) with 64% fucose,
R297 with 40% fucose,

C029-025 with 33% fucose,
R297 with 25% fucose.

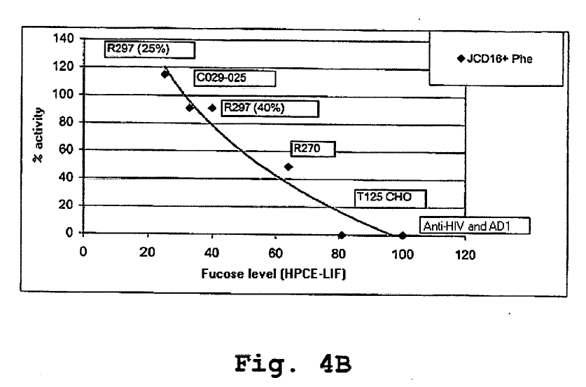

FIGS. 4A and 4B represent respectively the results obtained in the presence of target cells in test (4A) and in the absence of target cells but with the antibody preparations aggregated using a F(ab′)₂. Whatever the test used for determining the activity of the samples, a reduction in the functional activity of the antibodies vis-à-vis the CD16 is noted when the fucose level increases. An antibody having a fucose level of 100% such as AD1 even has a complete absence of activity vis-à-vis this receptor. This study makes it possible to confirm that the fucose level influences the binding of the antibody to the CD16. A totally fucosylated immunoglobulin at the level of the oligosaccharide of the asparagine in position 297 of the heavy chain prevents any interaction of the antibody with the CD16, thus does not result in its activation.

### Example 4

### Study of the Monitoring of the Stability of a Monoclonal Antibody

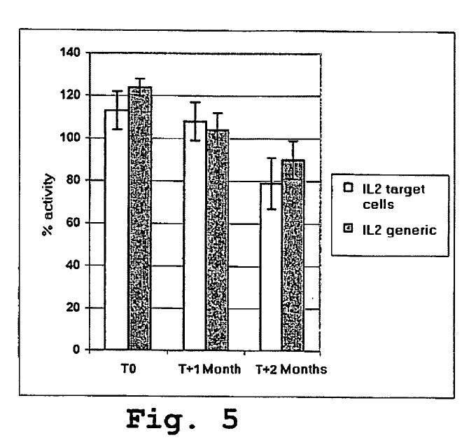

Another application of this test is the accelerated monitoring of the stability of a therapeutic preparation placed under denaturing conditions (cf. FIG. 5). This monitoring is carried out over several months by measuring the activity of the sample each month. This study was carried out on the sample No. 05-081 placed at 40° C. Its activity was tested at different times (T0, T+1 month and T+2 months) in the test with target cells but also in the test without target cells, also called a generic test. 100% activity is determined by means of a reference antibody (the research batch R297 for the test with target cells and the pilot batch C029-025 for the generic test).

This study makes it possible to detect a loss of activity of the sample when it is exposed to a high temperature. This loss is proportional to the duration of exposure of the sample to heat. This study also shows that similar results are obtained for both tests. These two tests can therefore be used for monitoring stability over time of preparations for therapeutic use.

### Example 5

### Comparison of the Specific Production of IL-2 by Antibodies Aggregated by Heat or by a F(ab)′ 2 Fragment Directed Against a (Fab)′2 IgG Fragment

The dose effect of anti-D monoclonal antibody preparations aggregated either by heat (20 minutes at 63° C.), or by a F(ab)′2 directed fragment against a (Fab)′₂ IgG fragment on the production of IL-2 by a Jurkat line genetically recombined to express CD16 was studied.

The aggregated monoclonal antibodies were incubated with the Jurkat CD16⁺ cells for 16 hours at 37° C. before dosing the IL-2 into the supernatants.

The results are given in Table 1 below:

TABLE 1

| Monoclonal Ab | Specific production of IL-2 in pg/ml | |
|---|---|---|
| Anti-D (ng/ml) | IgG aggregated by heat | IgG aggregated with F(ab)′₂ |
| 10 000 | 2965 | 8652 |
| 5000 | 897 | 8190 |
| 1000 | 102 | 856 |
| 500 | 22 | 171 |
| 0 | <12 | <12 |

Table 1 shows that a dose/effect relationship exists between the quantity of IL-2 secreted by the cells in the supernatant and the aggregated monoclonal antibody concentration present in the test, regardless of the method used for aggregating the monoclonal antibodies.

However, it is observed that the quantity of IL-2 secreted by the monoclonal antibodies aggregated by heat is lower than if the antibodies are aggregated by an IgG anti-F(ab′)₂.

### BIBLIOGRAPHY

1. Van Mirre et al. (2004). Monomeric IgG in intravenous Ig preparations is a functional antiagonist of FcgRII and FcgRIIIb. The journal of Immunology; 332:339
2. Farag S. S., Flinn I. W., Modali R., Lehman T. A., Young D., and Byrd J. C. (2004) Blood, vol. 103, n[.]4, 1472-1474.
3. Wu J., Edberg J. C, Redecha P. B., Bansal V., Guyre P. M., Coleman K., Salmon J. E., and Kimberly R. P. 1997, J.) Clin. Invest. Vol. 100, n[.]5, 1059-1070.
4. Shields R. L., Lai J., Keck R., O'Connell L: Y., Hong K., Meng Y. G., Weikert S. H. A., and Presta L. G.: (2002) J. Biol. Chem., vol. 277, n[.]30, 26733-26740.
5. Niwa R., Hatanaka S., Shoji-Hosaka E., Sakurada M., Kobayashi Y., -Uehara A., Yokoi H., Nakamura K., and Shitara K. (2004), Clinical Cancer Research, 10, 6248-6255.
6. Weiss A., Wiskocil R. L., and Stobo J. D. 1984, J. Immunol., vol. 133, n[.]1, 123-128.
7. Schneider U., Schwenk H. U., and Bornkamm G. (1977) Int. J. Cancer., vol. 19, n[.]5, 621-626.

1. Method for measuring the ability of an antibody preparation to activate an Fc receptor, comprising:
a) aggregating said antibodies with each other,
b) bringing cells expressing an Fc receptor into contact with said aggregated antibodies, and
c) measuring the reaction of said cells resulting from the activation of the Fc receptor of said cells by the Fc region of said antibodies.

2. Method according to claim 1, characterized in that said aggregation is carried out by means of an anti-IgG F(ab′)₂ fragment.

3. Method according to claim 2, characterized in that said anti-IgG F(ab′)₂ fragment is an anti-Fab or a anti-F(ab′)₂ directed against the antibody preparation tested.

4. Method according to claim 1, characterized in that said aggregation is an aggregation by heat.

5. Method according to claim 1, characterized in that said aggregation is carried out by cross-linking the Fab regions to each other or the heavy and light chains to each other.

6. Method according to claim 1, characterized in that said Fc receptor is selected from CD16 (FcγRIIIa and FcγRIIIb), CD32 (FcγRIIa and FcγRIIb), and CD64 (FcγRI).

7. Method according to claim 1, characterized in that said cells expressing said Fc receptor are transfected cells with the gene encoding said receptor.

8. Method according to claim 1, characterized in that said cells expressing said Fc receptor are Jurkat cells expressing CD16 and that this line is cultured in the presence of an aspecific activator of these cells such as PMA (Phorbol 12-Myristate 13-Acetate).

9. Method according to claim 1, characterized in that the measurement of the reaction of the cells resulting from the activation of the Fc receptor of said cells by the Fc region of said antibodies is a measurement of the quantity of at least one cytokine produced by the cells expressing said Fc receptor.

10. Method according to claim 9, characterized in that the measurement of at least one cytokine is effected by carrying out an assay of the mRNAs of said cytokines.

11. Method according to claim 10, characterized in that at least one cytokine selected from IL-1, IL-2, IL-3, IL-4, IL-5, IL-6, MCP-1, TNFalpha (TNFα) and IFNgamma (IFNγ) is quantified.

12. Method according to claim 11, characterized in that interleukin IL-2 is quantified.

13. Method according to claim 12, characterized in that the level of secreted interleukin IL-2 is correlated to an ADCC-type activity.

14. Method according to claim 1, characterized in that the measurement of the cell reaction is a measurement of the calcium influx, phosphorylation, transcription factors or apoptosis.

15. Method according to claim 1, for assessing the ability of a cell to produce a monoclonal antibody capable of interacting with the CD16 receptor.

16. Method according to claim 15, characterized in that said antibody-producing cell is chosen from the CHO, YB2/0, SP2/0, SP2/0-AG14, IR983F, the Namalwa human myeloma, PERC6 cells, the CHO lines, in particular CHO-K-1, CHO-Lec10, CHO-Lec1, CHO-Lec13, CHO Pro-5, CHO dhfr-, Wil-2, Jurkat, Vero, Molt-4, COS-7,293-HEK, BHK, K6H6, NS0, SP2/0-Ag 14 and P3X63Ag8.653.

17. Method according to claim 1, for assessing the effectiveness and the integrity of the Fc region of antibodies after one or more purification stages, or for monitoring the stability of a therapeutic preparation placed under denaturing conditions.

18. Method according to claim 1, for measuring the functionality of the Fc region of a antibody.

19. Method according to claim 1 for assessing the production of monoclonal antibodies by transgenic plants or transgenic mammals.

20. Method according to claim 1 for selecting effective antibodies for a therapeutic treatment.

21. Method according to claim 1 for selecting antibody compositions, the fucose content of which is less than 65%, and preferably less than 40%.

22. Method for preparing a monoclonal antibody composition capable of activating the CD16 (FcγRIII) receptor comprising the following steps:
a) obtaining monoclonal antibodies from a hybridoma, a heterohybridoma, or any animal, plant or human cell line transfected by means of one or more vectors in order to express said antibodies,
b) aggregating the antibodies obtained in step a) by an anti-IgG F(ab′)₂ fragment,
c) adding the antibodies obtained in step b) in a reaction mixture comprising:
a. effector cells comprising cells expressing the CD16 (FcγRIII) receptor,
b. (Phorbol 12-Myristate 13-Acetate).
d) measuring the quantity of at least one cytokine produced by the cell expressing CD16,
e) selecting the antibody composition(s) for which an increase is measured greater than 0.5, 1, 2, 5, 10, 100 or 500 times the quantity of cytokine measured with respect to the control in the absence of antibodies or in the presence of a given antibody as a negative reference.

23. Method according to claim 22, characterized in that the measurement of the quantity of at least one cytokine produced by the cell expressing CD16 is a measurement of the quantity of IL-2 produced by the cell expressing CD16.

24. Kit for the implementation of a biological test for measuring the activity of therapeutic antibodies comprising (i) means for aggregating said therapeutic antibodies, (ii) cells capable of expressing an Fc receptor, (iii) means for measuring the reaction of said cells capable of expressing an Fc receptor when the Fc receptor of said cells is activated by the Fc region of said antibodies, and (iv) the other elements necessary for the implementation of the method according to claim 1.

25. Kit for the implementation of a biological test for measuring the activity of therapeutic antibodies according to claim 24, characterized in that the means for measuring the reaction of said cells are means for quantification of at least one cytokine produced by said cells.

26. Kit for the implementation of a biological test for measuring the activity of therapeutic antibodies according to claim 25, characterized in that the means for quantifying at least one cytokine are means of assaying the mRNAs of said cytokines.

27. Kit for the implementation of a biological test for measuring the activity of therapeutic antibodies according to claim 24, characterized in that the means for measuring the reaction of said cells are means for measuring the calcium influx, phosphorylation, transcription factors or apoptosis.


SEQUENCE LISTING (4 sequences)

SEQ ID NO: 1 - DNA, 20 bp, Artificial Sequence
  Description of Artificial Sequence Synthetic primer
aacagtgcacctacttcaag

SEQ ID NO: 2 - DNA, 20 bp, Artificial Sequence
  Description of Artificial Sequence Synthetic primer
gttgagatgatgctttgaca

SEQ ID NO: 3 - DNA, 20 bp, Artificial Sequence
  Description of Artificial Sequence Synthetic primer
gggtcagaaggattcctatg

SEQ ID NO: 4 - DNA, 20 bp, Artificial Sequence
  Description of Artificial Sequence Synthetic primer
ggtctcaaacatgatctggg